(19) United States
(12) Patent Application Publication
(10) Pub. No.: US 2006/0241158 A1
(43) Pub. Date: Oct. 26, 2006

(54) Zolmitriptan crystal forms

(76) Inventors: Reuven Izsak, Jerusalem (IL); Ori Lerman, Givatayim (IL); Tamas Koltai, Natanya (IL); Judith Aronhime, Rehovot (IL); Michael Pinchasov, Rishon-Lezion (IL); Hagit Eisen-Nevo, Petah-Tikva (IL)

Correspondence Address: KENYON & KENYON LLP, ONE BROADWAY, NEW YORK, NY, 10004, US

(21) Appl. No.: 11/471,364

(22) Filed: Jun. 19, 2006

Related U.S. Application Data
(62) Division of application No. 11/284,773, filed on Nov. 21, 2005.
(60) Provisional application No. 60/629,649, filed on Nov. 19, 2004.
(60) Provisional application No. 60/631,916, filed on Nov. 30, 2004.
(60) Provisional application No. 60/681,672, filed on May 16, 2005.
(60) Provisional application No. 60/697,001, filed on Jul. 5, 2005.
(60) Provisional application No. 60/714,145, filed on Sep. 1, 2005.

(51) Int. Cl. A61K 31/422; C07D 413/02

(52) U.S. Cl. 514/376; 548/229

(57) ABSTRACT

The invention encompasses novel crystalline forms of zolmitriptan herein defined as Form B, D, C, E, F, G, H, I, J, K, M, N, O, P, Q, R, S, or Amorphous and to methods of making thereof. The invention also encompasses methods of making zolmitriptan crystalline Form A.

### RELATED APPLICATIONS

The present application claims the benefit of U.S. Provisional Application No. 60/629,649 filed Nov. 19, 2004; U.S. Provisional Application No. 60/631,916 filed Nov. 30, 2004; U.S. Provisional Application No. 60/681,672 filed May 16, 2005; U.S. Provisional Application No. 60/697,001 filed Jul. 5, 2005; and U.S. Provisional Application No. 60/714,145 filed on Sep. 1, 2005, which are incorporated herein by reference.

### FIELD OF INVENTION

The invention encompasses zolmitriptan crystal forms and methods of preparing the crystal forms. The invention also encompasses pharmaceutical compositions comprising zolmitriptan crystal forms and methods of treating migraine headache using the same.

### BACKGROUND OF THE INVENTION

Zolmitriptan has the chemical name (S)-4-{{3-[2-(dimethylaminoethyl]-1H-indol-5-yl]methyl]-2-oxazolidinone. Zolmitriptan is a selective 5-hydroxytryptamine 1B/1D (5-HT_{1B/1D}) receptor agonist. This receptor mediates vasoconstriction, and thus modifies blood flow to the carotid vascular bed. Agonists of the 5-HT_{1B/1D} receptor are therefore beneficial in the treatment (including prophylaxis) of disease conditions where vasoconstriction in the carotid vascular bed is indicated. Such conditions include migraine, cluster headache, and headache associated with vascular disorders, referred to collectively as “migraine.” Due to its agonist effect at the 5-HT receptor, zolmitriptan has been developed for the acute treatment of migraine.

U.S. Pat. No. 6,750,237 discloses a stable pharmaceutical formulation of zolmitriptan suitable for nasal administration, and the treatment of migraine using the nasal administration of zolmitriptan. Also disclosed is a method of preparing the zolmitriptan formulation by forming the citrate salt of zolmitriptan and then adding a buffer to the solution to bring the pH to a desired value.

U.S. Pat. No. 5,863,935 discloses heterocyclic compounds that act as antagonists of the 5-HT receptor. Example 2 discloses the preparation of (S)-N,N-dimethyl-2-[5-(2-oxo-1,3-oxazolidin-4-ylmethyl)-1H-indol-3-yl]ethylamine 0.9 isoproanolate hemihydrate.

U.S. Pat. No. 5,466,699 discloses indolyl compounds that act as antagonists of the 5-HT receptor. Examples 2 and 3 disclose the preparation of (S)-N,N-dimethyl-2-[5-(2-oxo-1,3-oxazolidin-4-ylmethyl)-1H-indol-3-yl]ethylamine 0.9 isoproanolate hemihydrate.

Polymorphism, the occurrence of different crystal forms, is a property of some molecules and molecular complexes. A single molecule may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for polymorphic forms of zolmitriptan.

### SUMMARY OF THE INVENTION

The invention encompasses novel solid states of zolmitriptan and methods of preparing these solid states and others. The invention also encompasses pharmaceutical compositions comprising solid states of zolmitriptan and methods of treating migraine headache using the compositions.

One embodiment of the invention encompasses a zolmitriptan crystal form characterized by X-ray powder diffraction peaks at 15.6, 19.5, 19.9, 22.2, and 24.5 degrees two-theta, ±0.2 degrees two-theta.

Another embodiment of the invention encompasses a zolmitriptan form characterized by X-ray powder diffraction peaks at 11.7, 14.0, 19.5, 23.0, and 23.2 degrees two-theta, ±0.2 degrees two-theta.

Another embodiment of the invention encompasses a zolmitriptan crystal form characterized by X-ray powder diffraction peaks at 11.8, 17.1, 18.3, 19.9, and 23.4 degrees two-theta, ±0.2 degrees two-theta.

Another embodiment of the invention encompasses a zolmitriptan crystal form characterized by X-ray powder diffraction peaks at 12.0, 18.4, 22.2, 22.4, and 23.7 degrees two-theta, ±0.2 degrees two-theta.

Another embodiment of the invention encompasses a zolmitriptan crystal form characterized by X-ray powder diffraction peaks at 11.6, 18.4, 21.2, 24.4 and 25.6 degrees two-theta, ±0.2 degrees two-theta.

Another embodiment of the invention encompasses a zolmitriptan crystal form characterized by X-ray powder diffraction peaks at 14.9, 17.0, 19.5, 21.9, and 24.2 degrees two-theta, ±0.2 degrees two-theta.

Another embodiment of the invention encompasses a zolmitriptan crystal form characterized by X-ray powder diffraction peaks at 15.1, 18.5, 21.5, 22.1, and 24.4 degrees two-theta, ±0.2 degrees two-theta.

Another embodiment of the invention encompasses a zolmitriptan crystal form characterized by X-ray powder diffraction peaks at 15.0, 18.3, 21.0, 21.9 and 25.6 degrees two-theta, ±0.2 degrees two-theta.

Another embodiment of the invention encompasses a zolmitriptan crystal form characterized by X-ray powder diffraction peaks at 11.3, 17.8, 19.8, 22.4, and 23.5 degrees two-theta, ±0.2 degrees two-theta.

Another embodiment of the invention encompasses a zolmitriptan crystal form characterized by X-ray powder diffraction peaks at 13.8, 15.1, 19.9, 23.9, and 25.6 degrees two-theta, ±0.2 degrees two-theta.

Another embodiment of the invention encompasses a zolmitriptan crystal form characterized by X-ray powder diffraction peaks at 15.3, 17.3, 20.0, 22.0, and 23.8 degrees two-theta, ±0.2 degrees two-theta.

Another embodiment of the invention encompasses a zolmitriptan crystal form characterized by X-ray powder diffraction peaks at 8.9, 16.8, 17.6, 19.9, and 26.2 degrees two-theta, ±0.2 degrees two-theta.

Another embodiment of the invention encompasses a zolmitriptan crystal form characterized by X-ray powder diffraction peaks at 12.3, 19.9, 22.9, 23.9, and 25.0 degrees two-theta, ±0.2 degrees two-theta.

Another embodiment of the invention encompasses zolmitriptan crystal form characterized by X-ray powder diffraction peaks at 8.7, 17.1, 17.8, 18.3, and 25.7 degrees two-theta, ±0.2 degrees two-theta.

Another embodiment of the invention encompasses a zolmitriptan crystal form characterized by X-ray powder diffraction peaks at 15.5, 18.0, 22.1 and 26.0 degrees two-theta, ±0.2 degrees two-theta.

Another embodiment of the invention encompasses a zolmitriptan crystal form characterized by X-ray powder diffraction peaks at 8.4, 14.6, 16.6, 22.7, and 23.7 degrees two-theta, ±0.2 degrees two-theta.

Another embodiment of the invention encompasses a zolmitriptan crystal form characterized by X-ray powder diffraction peaks at 18.1 and 27.2 degrees two-theta, ±0.2 degrees two-theta.

Another embodiment of the invention encompasses a zolmitriptan crystal form characterized by X-ray powder diffraction peaks at 8.8, 13.8, 17.5, 19.7, and 26.6 degrees two-theta, ±0.2 degrees two-theta.

Another embodiment of the invention encompasses a zolmitriptan crystal form characterized by X-ray powder diffraction peaks at 8.2, 12.1, 18.3, 19.8 and 25.1 degrees two-theta, ±0.2 degrees two-theta.

Another embodiment of the invention encompasses an amorphous form of zolmitriptan.

Another embodiment of the invention encompasses processes for preparing a crystalline form having X-ray powder diffraction peaks at 13.3, 13.9, 15.6, 17.1, 19.3, 22.1, 23.5 and 24.0 degrees two-theta, ±0.2 degrees two-theta, herein defined as form A.

These processes are crystallization, precipitation, slurry and drying.

Form A may be prepared by crystallization from a solvent selected from the group consisting of: 1-butanol, 2-butanol, methyl ethyl ketone, cyclopentanone, cyclohexanone, MIBK, butyl acetate, piperidine, pyridine, diethylamine, dioxane, dichloromethane and tetrahydrofuran. Preferably, the crystallization is from MIBK.

Form A may also be prepared by precipitation from a solvent/anti solvent pair selected from the group consisting of: DMSO/toluene, DMF/cyclohexane, ethanol/petrol ether 40-60 (P.E.), acetonitrile/cyclohexane, DMF/xylenes, acetonitrile/toluene, acetonitrile/chlorobenzene and acetonitrile/dichloromethane. Preferably, the precipitation is from DMSO/toluene.

Form A may also be prepared by slurrying one of crystal form D, G, K, Q and S in an acetone/water solution (20:80).

Form A may also be prepared by drying one of crystal form C, D, E, F, G, H, J, K, L, M, N, P, Q or S.

### BRIEF DESCRIPTION OF THE FIGURES

FIG. 1 illustrates the powder X-ray diffraction pattern for zolmitriptan Form B.

FIG. 2 illustrates the powder X-ray diffraction pattern for zolmitriptan Form C.

FIG. 3 illustrates the powder X-ray diffraction pattern for zolmitriptan Form D.

FIG. 4 illustrates the powder X-ray diffraction pattern for zolmitriptan Form E.

FIG. 5 illustrates the powder X-ray diffraction pattern for zolmitriptan Form F.

FIG. 6 illustrates the powder X-ray diffraction pattern for zolmitriptan Form G.

FIG. 6b illustrates the powder X-ray diffraction pattern for pure zolmitriptan Form G

FIG. 7 illustrates the powder X-ray diffraction pattern for zolmitriptan Form H.

FIG. 8 illustrates the powder X-ray diffraction pattern for zolmitriptan Form I.

FIG. 9 illustrates the powder X-ray diffraction pattern for zolmitriptan Form J.

FIG. 10 illustrates the powder X-ray diffraction pattern for zolmitriptan Form K.

FIG. 11 illustrates the powder X-ray diffraction pattern for zolmitriptan Form L.

FIG. 12 illustrates the powder X-ray diffraction pattern for zolmitriptan Form M.

FIG. 13 illustrates the powder X-ray diffraction pattern for zolmitriptan Form N.

FIG. 14 illustrates the powder X-ray diffraction pattern for zolmitriptan Form O.

FIG. 15 illustrates the powder X-ray diffraction pattern for zolmitriptanForm P.

FIG. 16 illustrates the powder X-ray diffraction pattern for zolmitriptan Form Q.

FIG. 17 illustrates the powder X-ray diffraction pattern for zolmitriptan Form R.

FIG. 18 illustrates the powder X-ray diffraction pattern for zolmitriptan Form S.

FIG. 19 illustrates the powder X-ray diffraction pattern for zolmitriptan Amorphous Form.

FIG. 20 illustrates the powder X-ray diffraction pattern for zolmitriptan Form A.

FIG. 21 illustrates the powder X-ray diffraction pattern for zolmitriptan Form T.

### DETAILED DESCRIPTION OF THE INVENTION

Zolmitriptan for use as a starting material in the methods of the invention may be prepared according to the disclosures of WO 91/18897, WO 97/06162, and U.S. Pat. No. 6,084,103. Without being bound to any particular theory, it is believed that the therapeutic activity of zolmitriptan for the treatment of migraine headache is attributed to its agonist effects at the 5-HT_{1B/1D} receptors on intracranial blood vessels (including the arterio-venous anastomoses) and sensory nerves of the trigeminal system which result in cranial vessel constriction and inhibition of pro-inflammatory neuropeptide release.

The term “spray drying” refers to processes involving breaking up liquid mixtures into small droplets (atomization) and rapidly removing solvent from the mixture. In a typical spray-drying apparatus, a heating drying gas provides a strong driving force for solvent evaporation in droplets. Spray-drying processes and equipment are described in Perry's Chemical Engineer's Handbook, pgs. 20-54 to 20-57 (Sixth Edition 1984).

By way of non-limiting example only, the typical spray-drying apparatus comprises a drying chamber, a method for atomizing a solvent-containing feed into the drying chamber, a source of heated drying gas that flows into the drying chamber to remove solvent from the atomized-solvent-containing feed, an outlet for the products of drying, and a cyclone (or other apparatus allowing collection of the product) located downstream of the drying chamber. Examples of such apparatuses include Niro Models PSD-1, PSD-2 and PSD-4 (Niro A/S, Soeborg, Denmark). In the cyclone or other collection apparatus, the particles produced during spray-drying are separated from the drying gas and evaporated solvent. A filter may also be used to separate and collect the particles produced by spray-drying.

The invention encompasses novel zolmitriptan solid states which may be characterized by X-Ray powder diffraction. In preferred embodiments of the invention, the zolmitriptan solid states described herein are substantially pure of zolmitriptan Form A. That is, the desired solid state of zolmitriptan has less than about 10% by weight of non-desired zolmitriptan Form A. Preferably, the desired solid state has less than about 5%, and more preferably less than about 1% by weight of zolmitriptan Form A. In an especially preferred embodiment, the zolmitriptan solid state is substantially pure of other solid states of zolmitriptan.

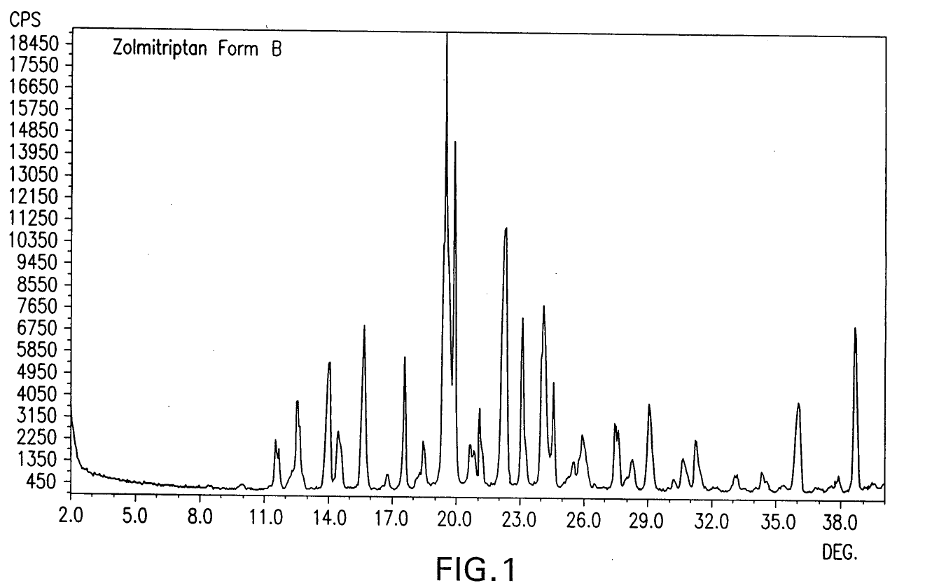

One embodiment of the invention encompasses zolmitriptan crystal form characterized by X-ray powder diffraction peaks at 15.6, 19.5, 19.9, 22.2, and 24.5 degrees two-theta, ±0.2 degrees two-theta. This form is denominated Form B. Form B may be characterized further by X-ray powder diffraction peaks at 20.6, 20.8, 24.1, 27.4, and 27.6 degrees two-theta, ±0.2 degrees two-theta. Form B may be substantially identified by FIG. 1. Form B may be produced as a solvate, preferably DMF solvate. Form B has at least one of a weight loss measured by TGA of about 7% by weight or a powder X-ray diffraction pattern.

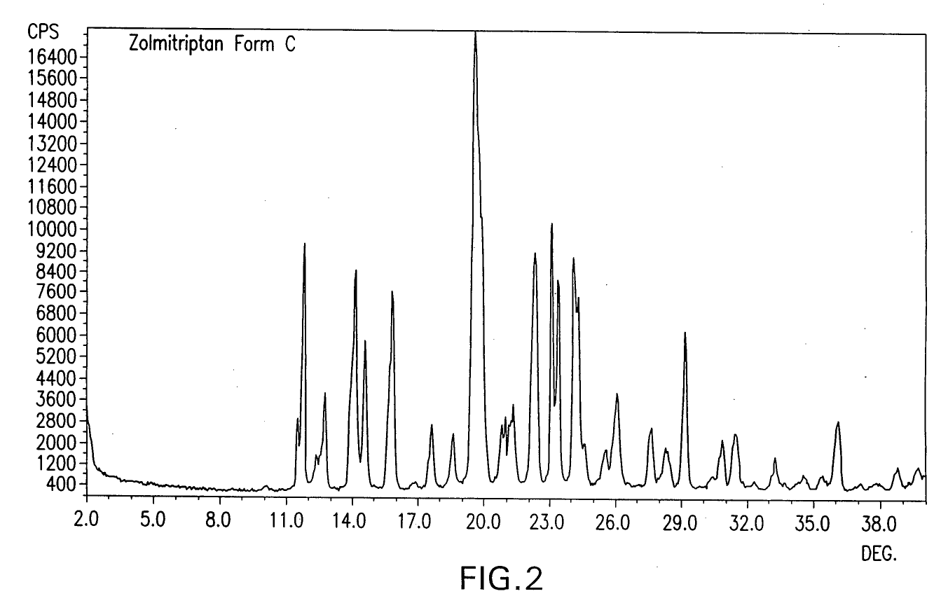

Another embodiment of the invention encompasses zolmitriptan crystal form characterized by X-ray powder diffraction peaks at 11.7, 14.0, 19.5, 23.0, and 23.2 degrees two-theta, ±0.2 degrees two-theta. This form is denominated Form C. Form C may be characterized further by X-ray powder diffraction peaks at 14.4, 15.7, 22.1, 24.0, and 24.2 degrees two-theta, ±0.2 degrees two-theta. Form C may be substantially identified by FIG. 2. Form C may be produced as a solvate, preferably ethanol solvate, preferably having about 4% water by weight. Form C has at least one of a weight loss measured by TGA of about 15% by weight or a powder X-ray diffraction pattern.

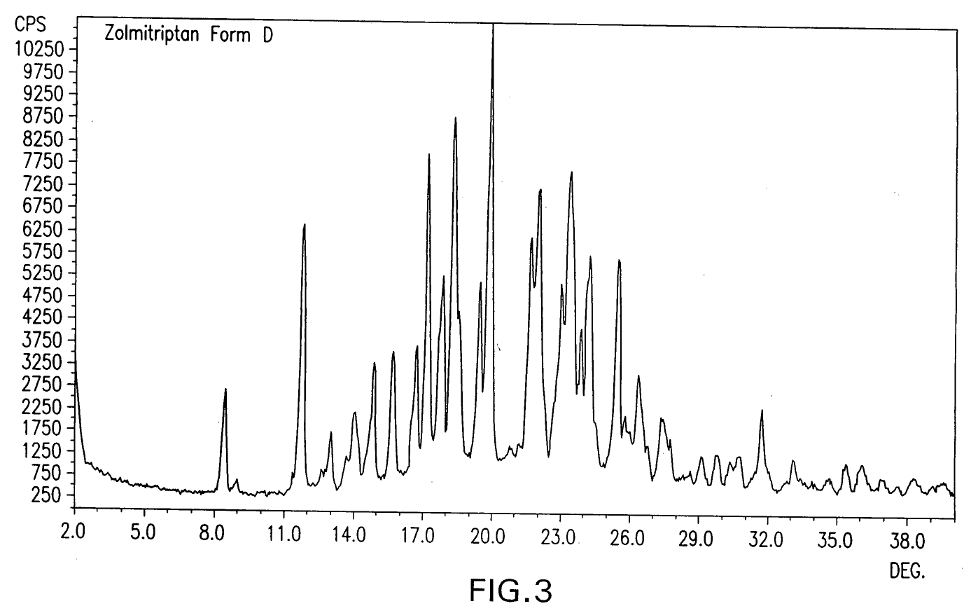

Another embodiment of the invention encompasses zolmitriptan crystal form characterized by X-ray powder diffraction peaks at 11.8, 17.1, 18.3, 19.9, and 23.4 degrees two-theta, ±0.2 degrees two-theta. This form is denominated Form D. Form D may be characterized further by X-ray powder diffraction peaks at 17.8, 19.4, 22.0, 24.2, and 25.4 degrees two-theta, ±0.2 degrees two-theta. Form D may be substantially identified by FIG. 3. Form D may be produced as a solvate, preferably 2-butanol or 1,3-dioxane solvate, preferably having about 1% water by weight. Form D has at least one of a weight loss measured by TGA of about 4% to 20% by weight or a powder X-ray diffraction pattern.

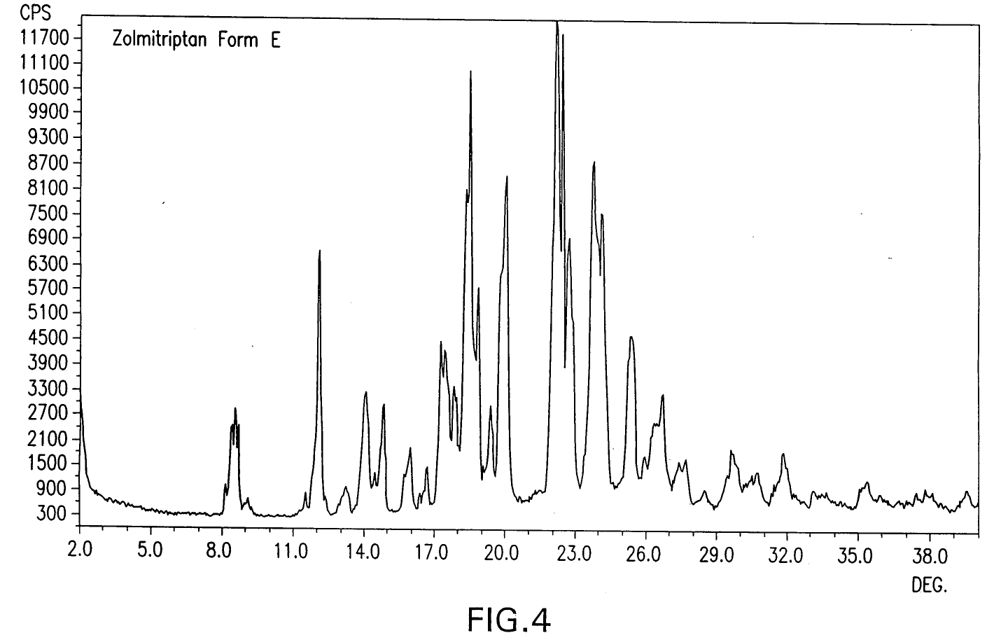

Another embodiment of the invention encompasses zolmitriptan crystal form characterized by X-ray powder diffraction peaks at 12.0, 18.4, 22.2, 22.4, and 23.7 degrees two-theta, ±0.2 degrees two-theta. This form is denominated Form E. Form E may be characterized further by X-ray powder diffraction peaks at 17.2, 20.0, 22.7, 24.1, and 25.3 degrees two-theta, ±0.2 degrees two-theta. Form E may be substantially identified by FIG. 4. Form E may be produced as a solvate, preferably 1-butanol solvate, preferably having about 1% water by weight. Form E has at least one of a weight loss measured by TGA of about 14% by weight or a powder X-ray diffraction pattern.

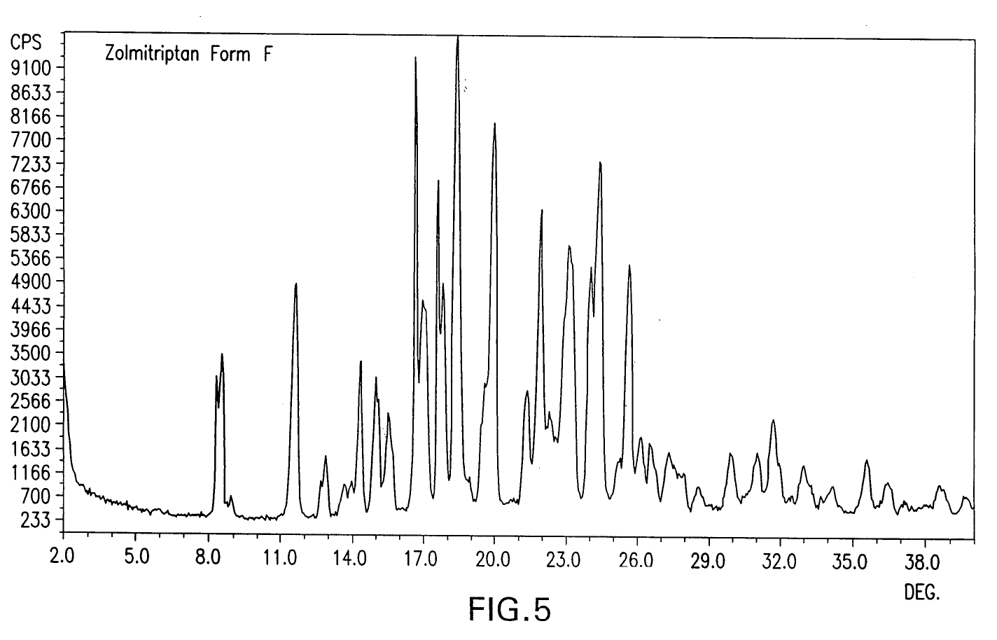

Another embodiment of the invention encompasses zolmitriptan crystal form characterized by X-ray powder diffraction peaks at 11.6, 18.4, 21.2, 24.4 and 25.6 degrees two-theta, ±0.2 degrees two-theta. This form is denominated Form F. Form F may be characterized further by X-ray powder diffraction peaks at 16.6, 17.6, 19.9, 21.9 and 23.1 degrees two-theta, ±0.2 degrees two-theta. Form F may be substantially identified by FIG. 5. Form F may be produced as a solvate, preferably isobutanol solvate, preferably having about 1% water by weight. Form F has at least one of a weight loss measured by TGA of about 12% by weight or a powder X-ray diffraction pattern.

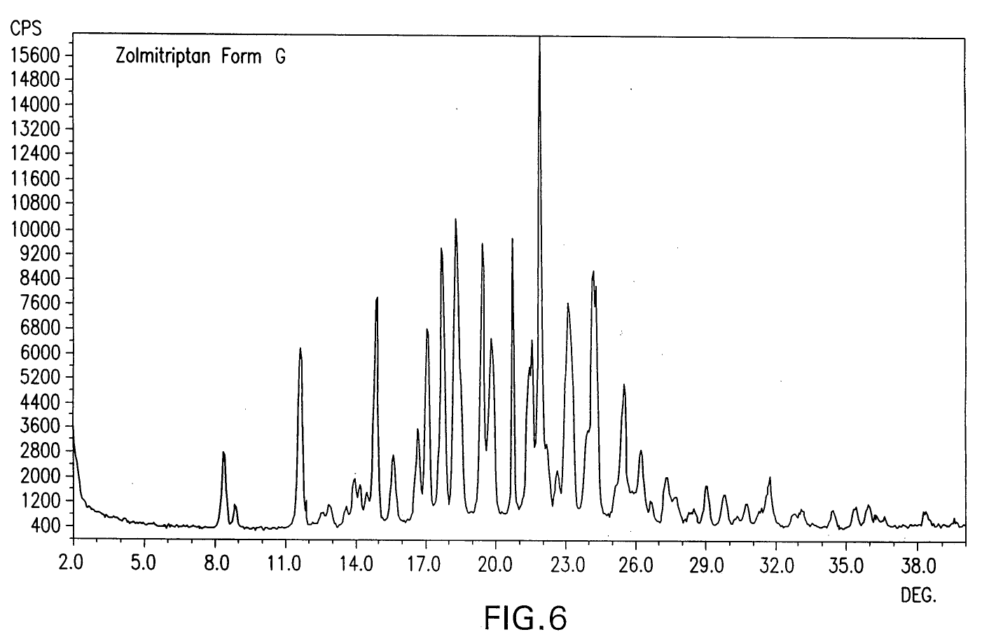

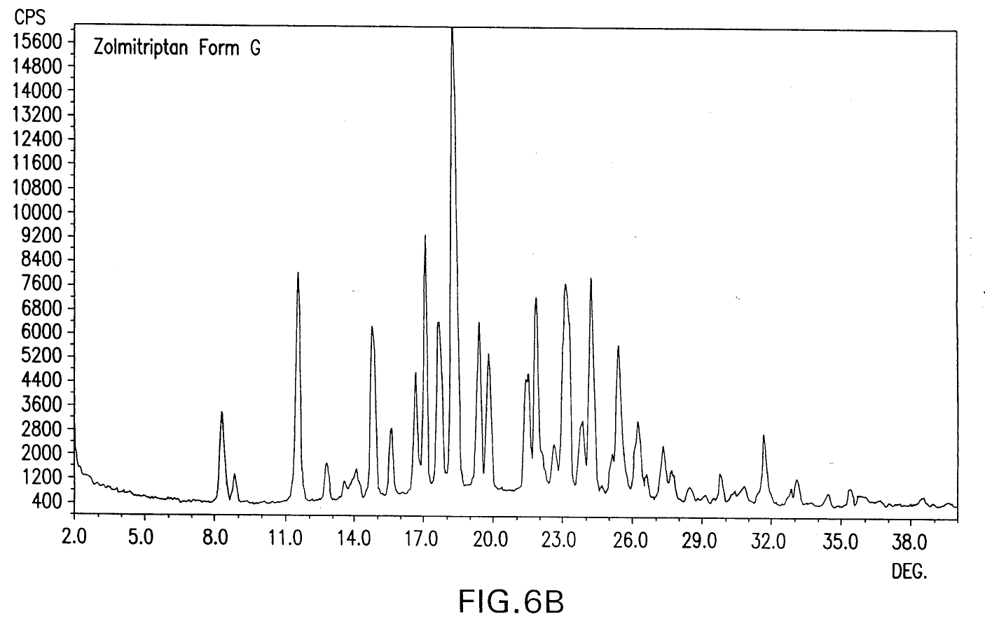

Another embodiment of the invention encompasses zolmitriptan crystal form characterized by X-ray powder diffraction peaks at 14.9, 17.0, 19.5, 21.9, and 24.2 degrees two-theta, ±0.2 degrees two-theta. This form is denominated Form G. Form G may be characterized further by X-ray powder diffraction peaks at 11.6, 17.6, 18.3 and 23.1 degrees two-theta, ±0.2 degrees two-theta. Form G may be substantially identified by FIG. 6. Form G may be produced as a solvate, preferably THF solvate, preferably having about 0.5% water by weight. Form G has at least one of a weight loss measured by TGA of about 18% by weight or a powder X-ray diffraction pattern.

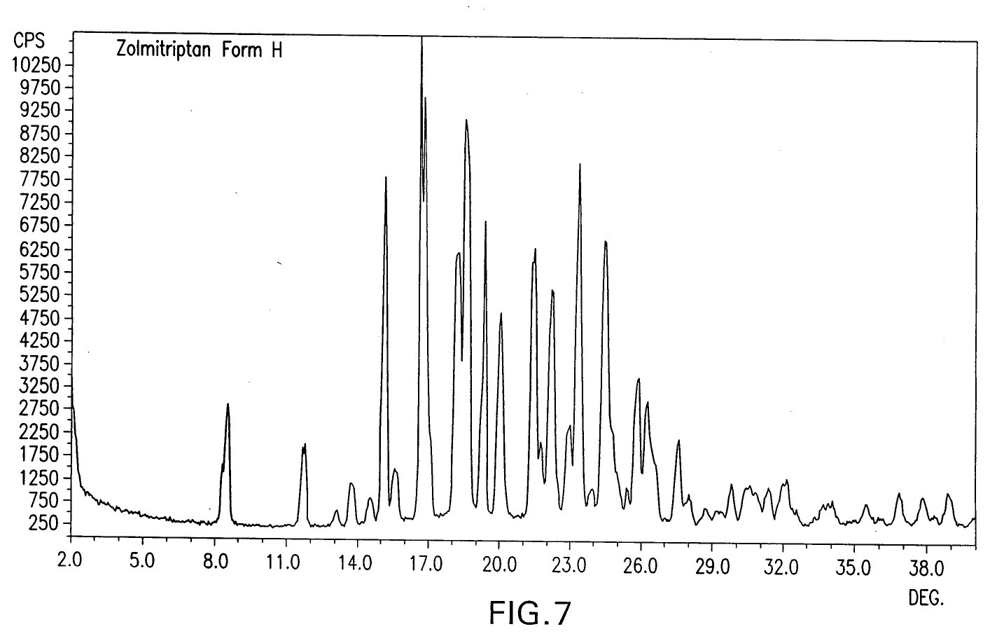

Another embodiment of the invention encompasses zolmitriptan crystal form characterized by X-ray powder diffraction peaks at 15.1, 18.5, 21.5, 22.1, and 24.4 degrees two-theta, ±0.2 degrees two-theta. This form is denominated Form H. Form H may be characterized further by X-ray powder diffraction peaks at 16.6, 18.2, 19.3, 20.0, and 23.3 degrees two-theta, ±0.2 degrees two-theta. Form H may be substantially identified by FIG. 7. Form H may be produced as a solvate, preferably cyclohexanone solvate, preferably having about 0.4% water by weight. Form H has at least one of a weight loss measured by TGA of about 5% by weight or a powder X-ray diffraction pattern.

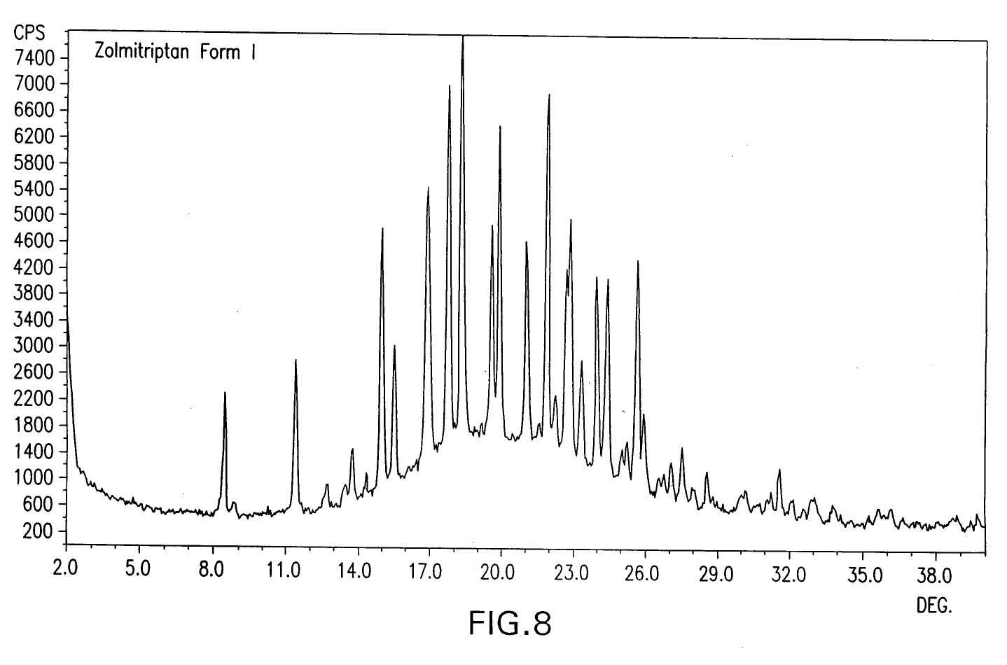

Another embodiment of the invention encompasses zolmitriptan crystal form characterized by X-ray powder diffraction peaks at 15.0, 18.3, 21.0, 21.9 and 25.6 degrees two-theta, ±0.2 degrees two-theta. This form is denominated Form I. Form I may be characterized further by X-ray powder diffraction peaks at 16.9, 17.7, 19.8, 22.9, and 24.4 degrees two-theta, ±0.2 degrees two-theta. Form I may be substantially identified by FIG. 8. Form I may be produced as a solvate, preferably 1,4-dioxane solvate, preferably having about 0.4% to 0.8% water by weight. Form I has at least one of a weight loss measured by TGA of about 6% to 43% by weight or a powder X-ray diffraction pattern.

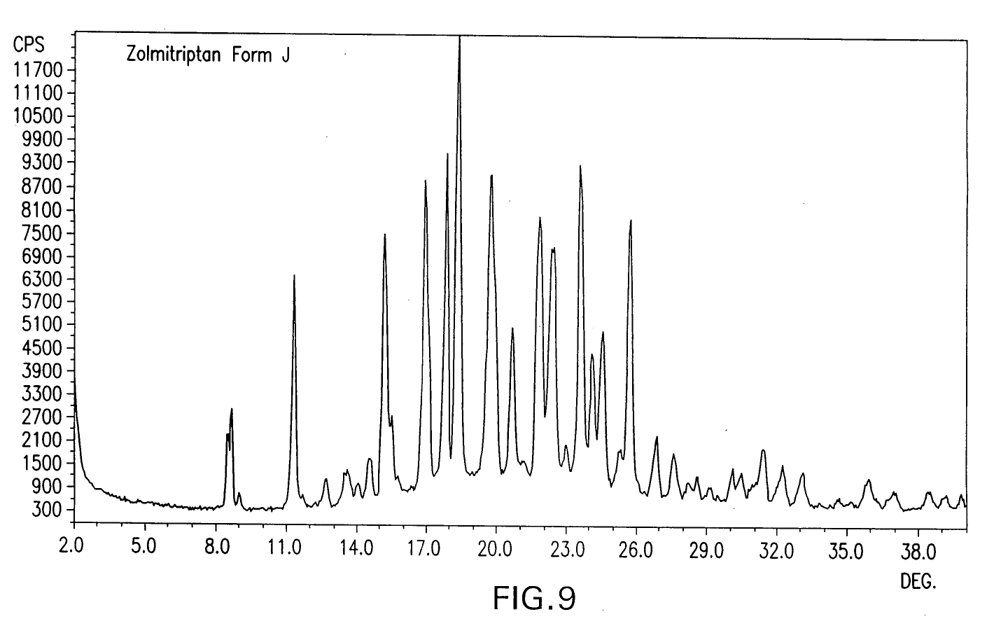

Another embodiment of the invention encompasses zolmitriptan crystal form characterized by X-ray powder diffraction peaks at 11.3, 17.8, 19.8, 22.4, and 23.5 degrees two-theta, ±0.2 degrees two-theta. This form is denominated Form J. Form J may be characterized further by X-ray powder diffraction peaks at 15.2, 16.9, 18.3, 19.6, 21.8, 22.1, 23.3 and 25.7 degrees two-theta, ±0.2 degrees two-theta. Form J may be substantially identified by FIG. 9. Form J may be produced as a solvate, preferably piperidine solvate, preferably having about 0.4% water by weight. Form J has at least one of a weight loss measured by TGA of about 10% by weight or a powder X-ray diffraction pattern.

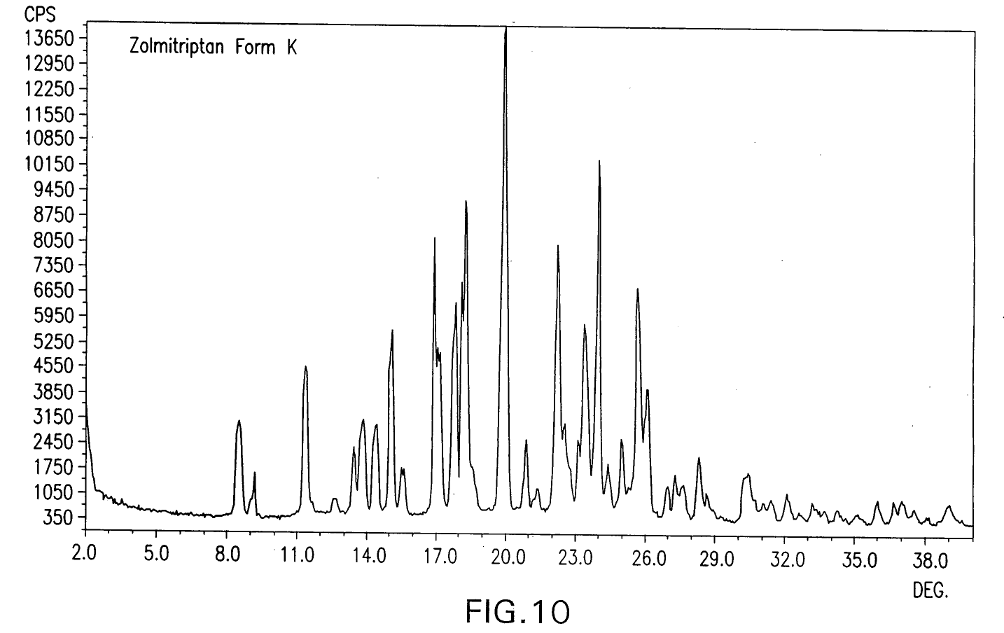

Another embodiment of the invention encompasses zolmitriptan crystal form characterized by X-ray powder diffraction peaks at 13.8, 15.1, 19.9, 23.9, and 25.6 degrees two-theta, ±0.2 degrees two-theta. This form is denominated Form K. Form K may be characterized further by X-ray powder diffraction peaks at 11.3, 17.8, 18.2, 19.3, 22.1, and 23.3 degrees two-theta, ±0.2 degrees two-theta. Form K may be substantially identified by FIG. 10. Form K may be produced as a solvate, preferably methanol, ethanol, dimethylforamide, or acetonitrile solvate, preferably having about 0.1% to 0.4% water by weight. Form K has at least one of a weight loss measured by TGA of about 11-21% by weight or a powder X-ray diffraction pattern.

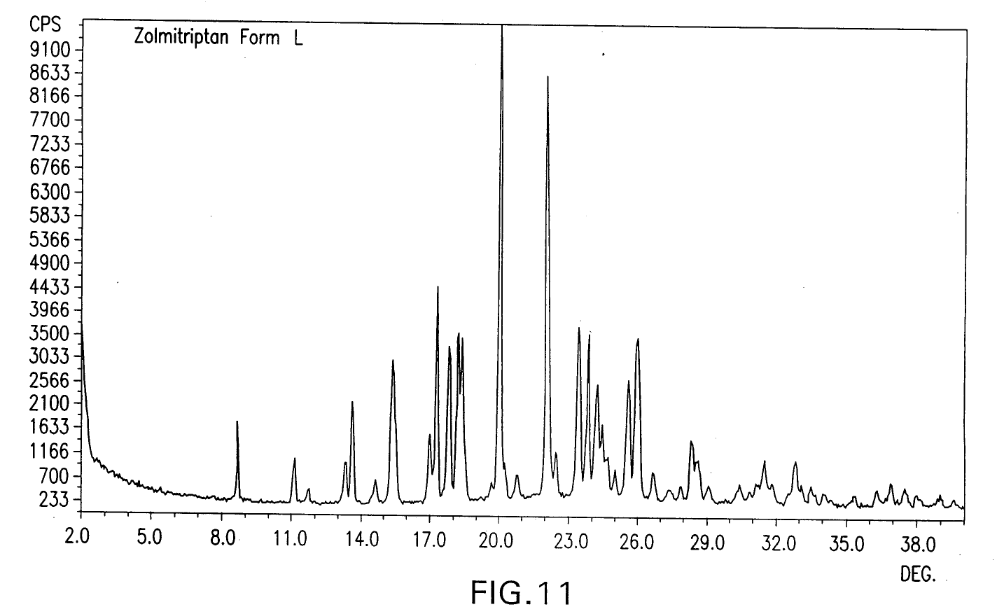

Another embodiment of the invention encompasses zolmitriptan crystal form characterized by X-ray powder diffraction peaks at 15.3, 17.3, 20.0, 22.0, and 23.8 degrees two-theta, ±0.2 degrees two-theta. This form is denominated Form L. Form L may be characterized further by X-ray powder diffraction peaks at 13.6, 17.8, 23.4, 25.5, and 25.9 degrees two-theta, ±0.2 degrees two-theta. Form L may be substantially identified by FIG. 11. Form L may be produced as a solvate, preferably acetonitrile solvate, preferably having about 0.1% water by weight. Form L has at least one of a weight loss measured by TGA of about 20% by weight or a powder X-ray diffraction pattern.

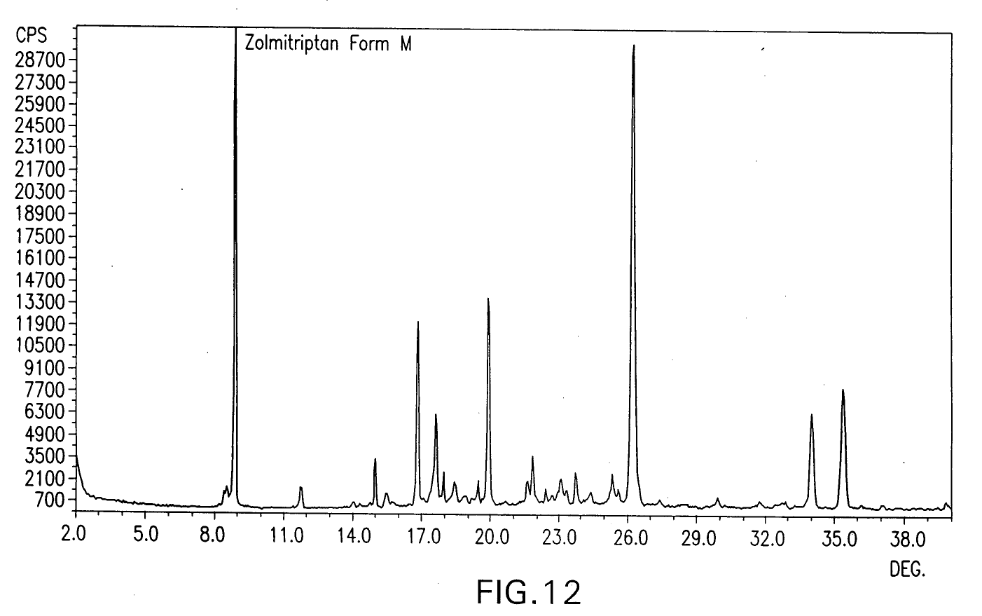

Another embodiment of the invention encompasses zolmitriptan crystal form characterized by X-ray powder diffraction peaks at 8.9, 16.8, 17.6, 19.9, and 26.2 degrees two-theta, ±0.2 degrees two-theta. This form is denominated Form M. Form M may be substantially identified by FIG. 12. Form M may be produced as a solvate, preferably cyclopentanone solvate, preferably having about 0.2% water by weight. Form M has at least one of a weight loss measured by TGA of about 10% by weight or a powder X-ray diffraction pattern.

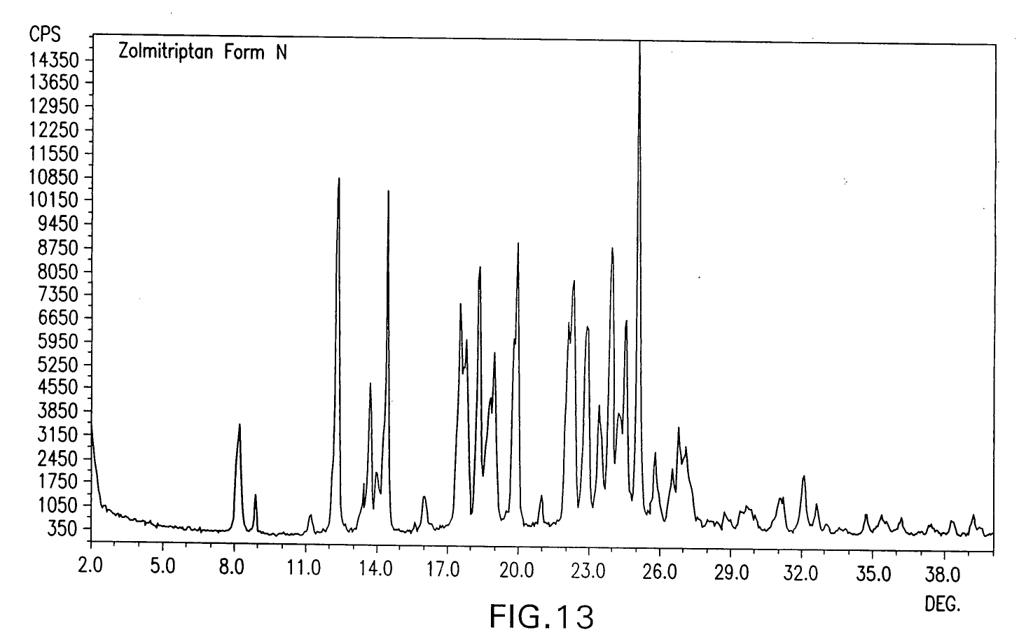

Another embodiment of the invention encompasses zolmitriptan crystal form characterized by X-ray powder diffraction peaks at 12.3, 19.9, 22.9, 23.9, and 25.0 degrees two-theta, ±0.2 degrees two-theta. This form is denominated Form N. Form N may be characterized further by X-ray powder diffraction peaks at 14.4, 17.5, 18.3, 18.9, and 22.2 degrees two-theta, ±0.2 degrees two-theta. Form N may be substantially identified by FIG. 13. Form N may be produced as a solvate, preferably methyl ethyl ketone solvate, preferably having about 0.3% water by weight. Form N has at least one of a weight loss measured by TGA of about 10% by weight or a powder X-ray diffraction pattern.

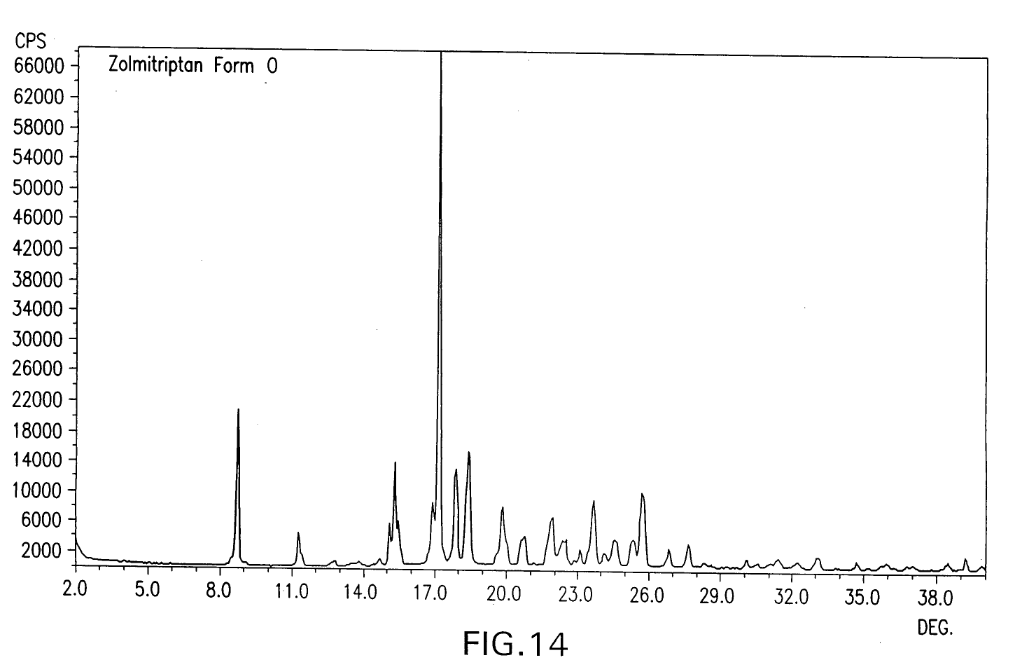

Another embodiment of the invention encompasses zolmitriptan crystal form characterized by X-ray powder diffraction peaks at 8.7, 17.1, 17.8, 18.3, and 25.7 degrees two-theta, ±0.2 degrees two-theta. This form is denominated Form O. Form O may be characterized further by X-ray powder diffraction peaks at 19.8, 23.6 degrees two-theta, ±0.2 degrees two-theta. Form O may be substantially identified by FIG. 14. Form O may be produced as a solvate, preferably piperidine solvate, preferably having about 1% water by weight. Form O has at least one of a weight loss measured by TGA of about 4% by weight or a powder X-ray diffraction pattern.

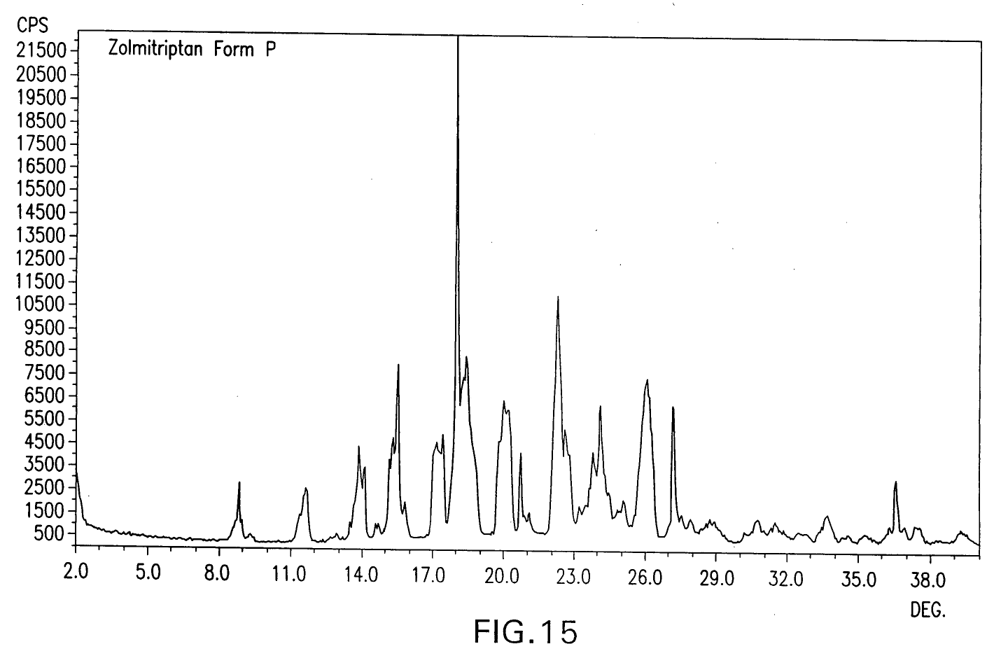

Another embodiment of the invention encompasses zolmitriptan crystal form characterized by X-ray powder diffraction peaks at 15.5, 18.0, 22.1 and 26.0 degrees two-theta, ±0.2 degrees two-theta. This form is denominated Form P. Form P may be substantially identified by FIG. 15. Form P may be produced as a solvate, preferably pyridine solvate, preferably having about 1% water by weight. Form P has at least one of a weight loss measured by TGA of about 10% by weight or a powder X-ray diffraction pattern.

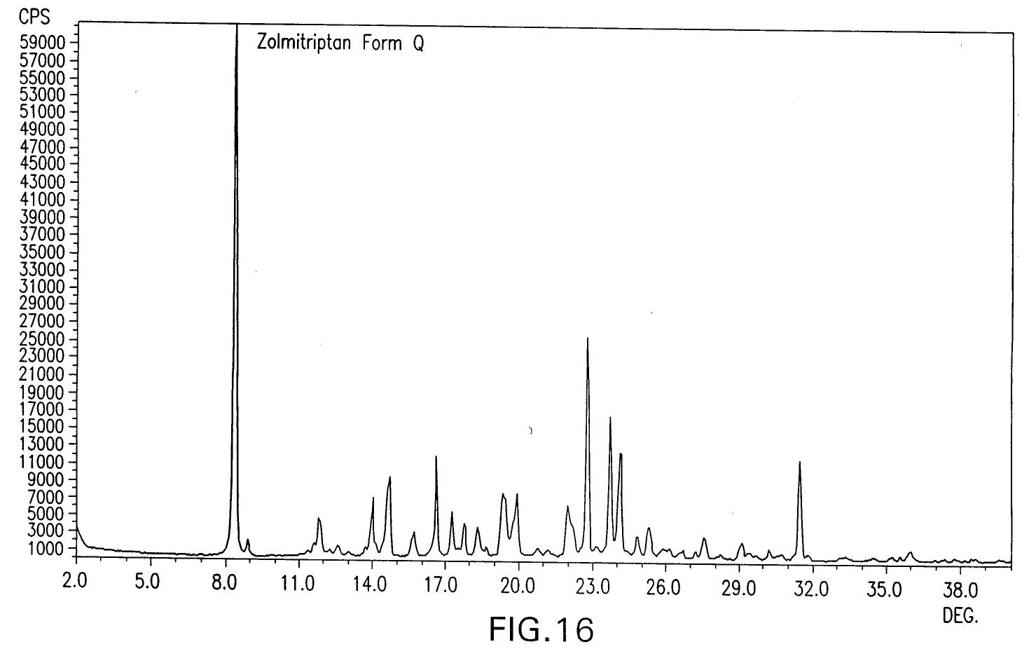

Another embodiment of the invention encompasses zolmitriptan crystal form characterized by X-ray powder diffraction peaks at 8.4, 14.6, 16.6, 22.7, and 23.7 degrees two-theta, ±0.2 degrees two-theta. This form is denominated Form Q. Form Q may be characterized further by X-ray powder diffraction peaks at 14.0, 19.3 degrees two-theta, ±0.2 degrees two-theta. Form Q may be substantially identified by FIG. 16. Form Q may be produced as a solvate, preferably diethylamine solvate, preferably having about 0.5% water by weight. Form has at least one of a weight loss measured by TGA of about 3% to about 12% by weight or a powder X-ray diffraction pattern.

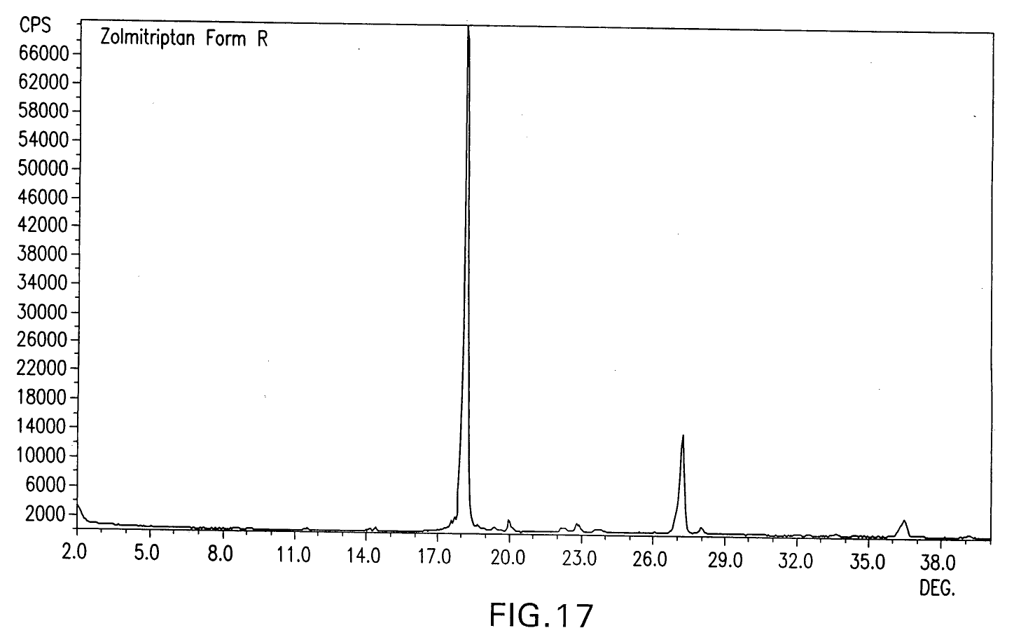

Another embodiment of the invention encompasses zolmitriptan crystal form characterized by X-ray powder diffraction peaks at 18.1 and 27.2 degrees two-theta, ±0.2 degrees two-theta. This form is denominated Form R. Form R may be substantially identified by FIG. 17. Form R may be produced as a solvate, preferably dichloromethane solvate, preferably having about 3% water by weight. Form R has at least one of a weight loss measured by TGA of about 18% by weight or by a powder X-ray diffraction pattern.

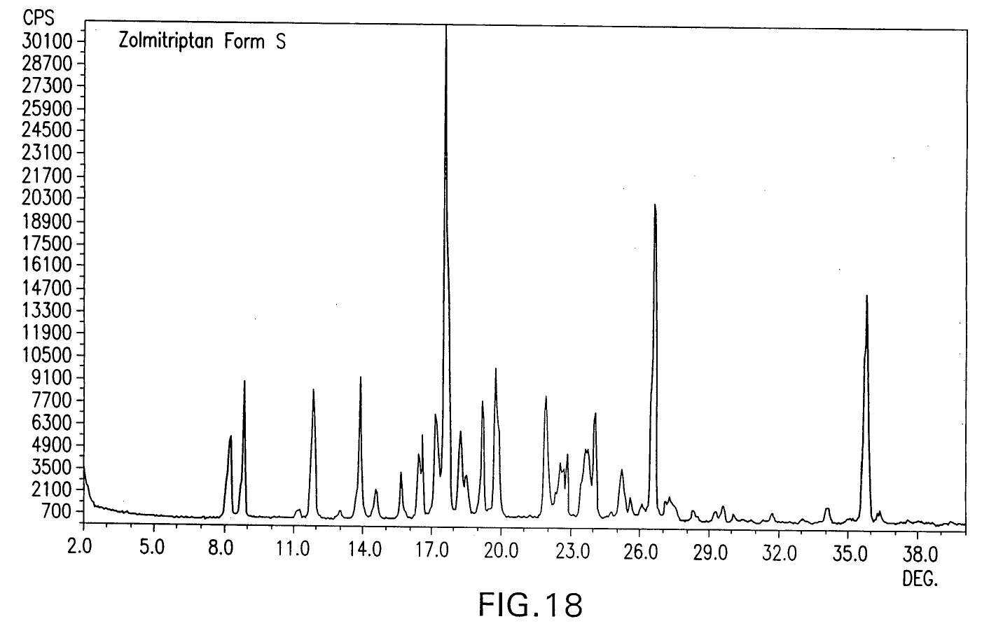

Another embodiment of the invention encompasses zolmitriptan crystal form characterized by X-ray powder diffraction peaks at 8.8, 13.8, 17.5, 19.7, and 26.6 degrees two-theta, ±0.2 degrees two-theta. This form is denominated Form S. Form S may be characterized further by X-ray powder diffraction peaks at 11.8, 21.9, 24.0 degrees two-theta, ±0.2 degrees two-theta. Form S may be substantially identified by FIG. 18.

Form S may be produced as a solvate, preferably butylacetate or n-butanol solvate, preferably having about 0.1% to 0.4% water by weight. Form S has at least one of a weight loss measured by TGA of about 10%-20% by weight or a powder X-ray diffraction pattern.

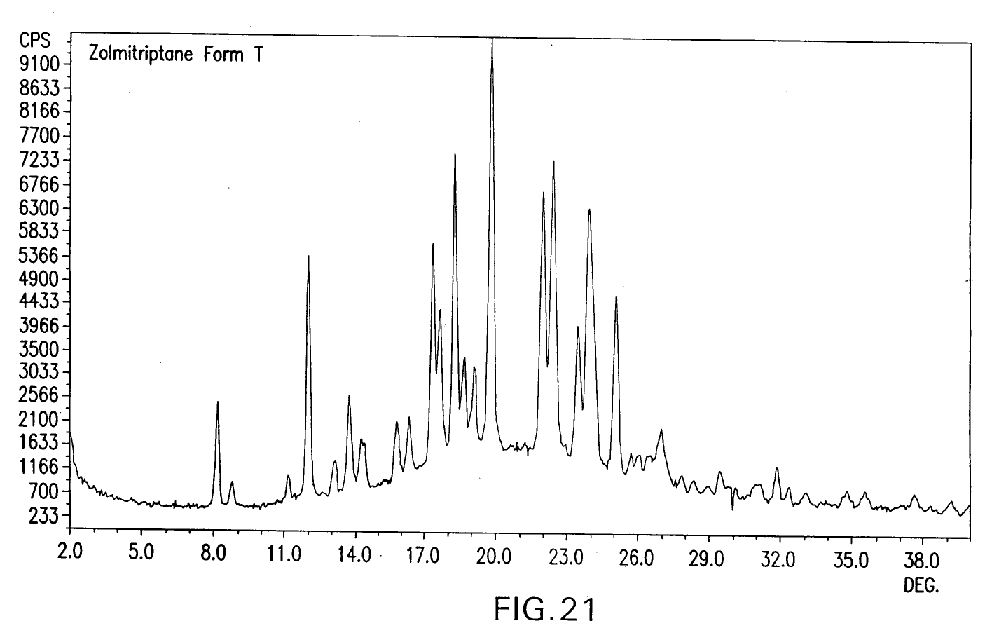

Another embodiment of the invention encompasses zolmitriptan crystal form characterized by X-ray powder diffraction peaks at 8.2, 12.1, 18.3, 19.8, and 25.1 degrees two-theta, ±0.2 degrees two-theta. This form is denominated Form T. Form T may be further characterized by X-ray powder diffraction peaks at 13.8, 17.3, 22.0, 22.5, 24.0 degrees two-theta, ±0.2 degrees two-theta. Form T may be substantially identified by FIG. 21. Form T may be produced as a solvate, preferably ethyl-acetate solvate, preferably having about 1% to 3% water by weight. Form T has at least one of a weight loss measured by TGA of about 4%-15% by weight or by a powder X-ray diffraction pattern.

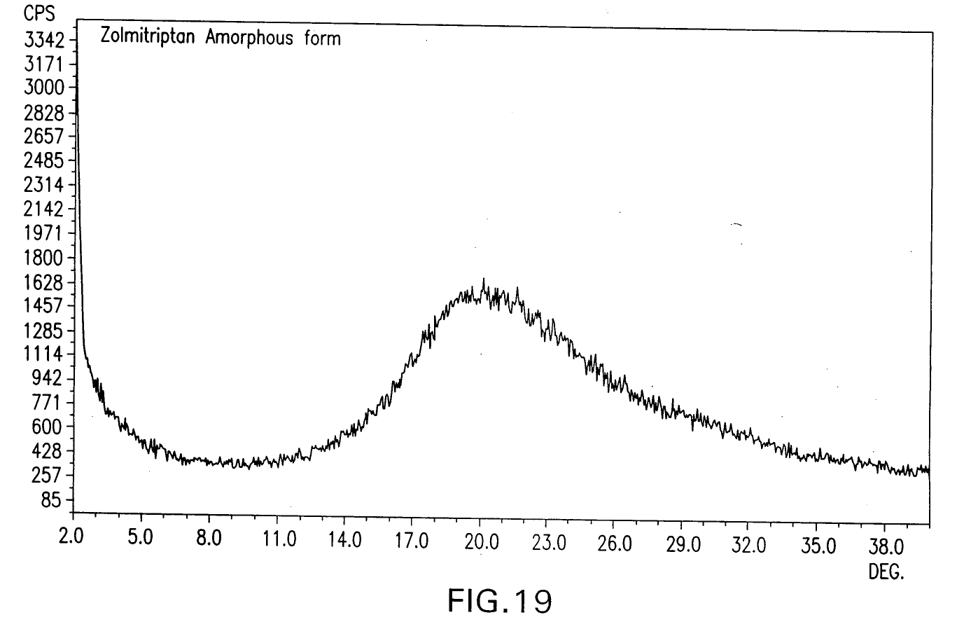

Another embodiment of the invention encompasses zolmitriptan amorphous form. Amorphous Form may be substantially identified by FIG. 19. Preferably, zolmitriptan amorphous form has about 0% to about 3% water by weight. Amorphous form may also be characterized by at least one of a weight loss measured by TGA of about 0% to about 3% by weight or by a powder X-ray diffraction pattern.

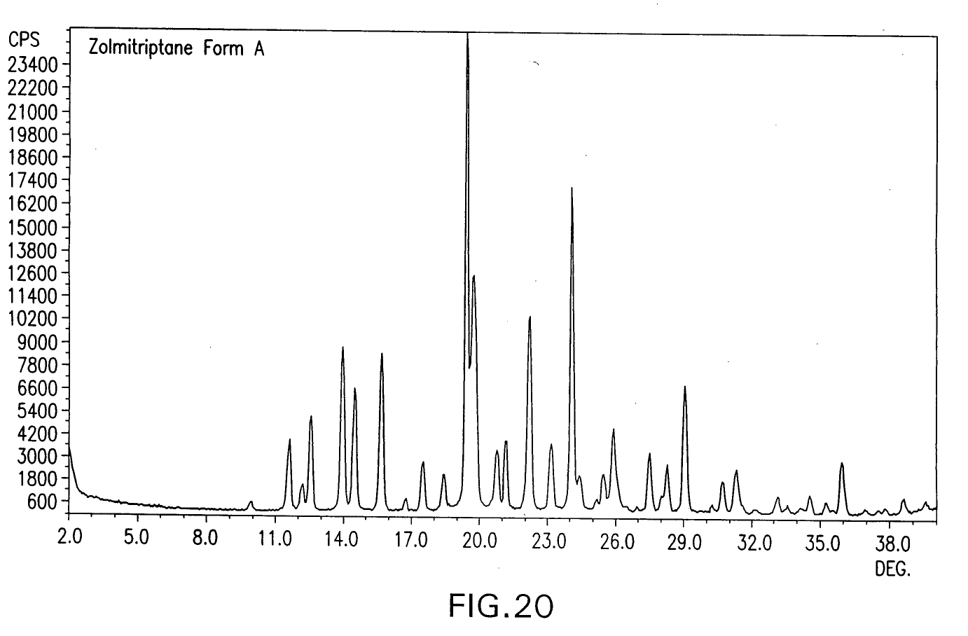

The invention also encompasses methods of preparing crystal form characterized by X-ray powder diffraction peaks at 13.3, 13.9, 15.6, 17.1, 19.3, 22.1, 23.5 and 24.0 degrees two-theta, ±0.2 degrees two-theta. This form is denominated Form A. Form A may be characterized further by X-ray powder diffraction peaks at 11.6, 12.5, 14.4, 19.7, and 29.0 degrees two-theta, ±0.2 degrees two-theta. Form A may be substantially identified by FIG. 20. Preferably, zolmitriptan Form A is an anhydrous crystal that has at least one of a weight loss measurement by TGA of about 0.2% by weight or a powder X-ray diffraction pattern.

In addition to other advantages posed by polymorphic forms, forms D, F, G, H, I, J, K, L, M, N, O, P, S, T and the amorphous form are particularly advantageous since they support pressing, and not transform to other crystal form.

As demonstrated in Table 1—pressing 100 mg one of the above zolmitriptan crystal form with approximately 1300 psi during 2 minutes, results in obtaining the same starting crystal form.

TABLE 1
Transformation by pressing

| Crystal Form before pressing | Crystal Form after pressing |
|---|---|
| Form D | Form D |
| Form F | Form F |
| Form G | Form G |
| Form H | Form H |
| Form I | Form I |
| Form J | Form J |
| Form K | Form K |
| Form L | Form L |
| Form M | Form M |
| Form N | Form N |
| Form O | Form O |
| Form P | Form P |
| Form S | Form S |
| Form T | Form T |
| Amorphous form | Amorphous Form |

The invention also encompasses a first method of preparing zolmitriptan crystal forms comprising: providing a mixture of zolmitriptan in a solvent selected from the group consisting of a C₁-C₄ alcohol, a C₃-C₇ ketone, a C₃-C₇ ester, amine, dioxane, dichloromethane and tetrahydrofuran; heating the mixture to a temperature of from about 40° C. to about 140° C.; maintaining the mixture at that temperature for about 10 minutes; cooling the mixture to a temperature of from about 0° C. to about 25° C. to obtain a precipitate and recovering the precipitate that is at least one of zolmitriptan Form A, D, E, F, G, H, I, M, N, O, P, Q, R, S or amorphous form.

Preferably, the solvent used to form the mixture is selected from the group consisting of: 1-butanol, 2-butanol, methyl ethyl ketone, cyclopentanone, cyclohexanone, MIBK, butyl acetate, piperidine, pyridine, diethylamine, dioxane, dichloromethane and tetrahydrofuran.

Preferably, the mixture of zolmitriptan and solvent is heated to about the lower of the reflux temperature of the solvent or 125° C.

Preferably, the mixture is cooled to a temperature of about 4° C.

The resulting precipitate may be recovered by any method commonly known in the art. Optionally, the method may further comprise drying the precipitate on a funnel.

Form A may be prepared by crystallization from a solvent selected from the group consisting of: 1-butanol, 2-butanol, methyl ethyl ketone, cyclopentanone, cyclohexanone, MIBK, butyl acetate, piperidine, pyridine, diethylamine, dioxane, dichloromethane and tetrahydrofuran. Preferably, the crystallization is from MIBK.

In a preferred embodiment, the present invention provides a method of preparing zolmitriptan crystal form A comprising: providing a mixture of zolmitriptan in MIBK; heating the mixture to a temperature of from about 60° C. to about 100° C.; maintaining the mixture for a period of about 10 minutes; cooling the mixture to a temperature of from about 0° C. to about 25° C. to obtain a precipitate and recovering Form A.

Preferably, the mixture is heated to a temperature of about 1 00° C.

Preferably, the mixture is gradually cooled to a temperature of about 4° C.

Form A may be recovered by any method known in art, such as filtration to dryness on a funnel, preferably for 30 minutes.

A second method of preparing zolmitriptan crystal forms encompasses providing a mixture of zolmitriptan in a solvent by heating to a temperature of from about 40° C. to about 140° C.; adding an anti-solvent to obtain a precipitate; maintaining the mixture for about 10 minutes; cooling the mixture to about 0° C. to about 25° C., and recovering the precipitate that is at least one of zolmitriptan Form A, B, C, J, K, L, P, amorphous form or a mixture thereof.

Preferably, the solvent/anti solvent pair used to induce precipitation of zolmitriptan crystal forms is selected from the group consisting of: DMSO/toluene, DMF/cyclohexane, ethanol/petrol ether 40-60 (P.E.), acetonitrile/cyclohexane, DMF/xylenes, acetonitrile/toluene, acetonitrile/chlorobenzene and acetonitrile/dichloromethane.

Preferably, the mixture of zolmitriptan and solvent is heated to about the lower of the reflux temperature of the solvent or 100° C.

Preferably, the mixture is cooled to a temperature of about 4° C.

The precipitate may be recovered by any method commonly known in the art. Optionally, the process may further comprise drying the precipitate on a funnel.

Form A may be prepared by precipitation from a solvent/anti solvent pair selected from the group consisting of: DMSO/toluene, DMF/cyclohexane, ethanol/petrol ether 40-60 (P.E.), acetonitrile/cyclohexane, DMF/xylenes, acetonitrile/toluene, acetonitrile/chlorobenzene and acetonitrile/dichloromethane. Preferably, the precipitation is from DMSO/toluene.

In a preferred embodiment the present invention provides a method of preparing zolmitriptan crystal form A comprising: providing a mixture of zolmitriptan in DMSO at a temperature of from about 40° C. to about 140° C.; adding toluene to the mixture; maintaining the mixture for about 10 minutes; cooling the mixture to a temperature of from about 0° C. to about 25° C. to obtain a precipitate and recovering Form A.

Preferably, the mixture is heated to a temperature of about 100° C.

Preferably, the mixture is gradually cooled to a temperature of about 4° C.

Form A may be recovered by any method known in art, such as filtration to dryness on a funnel, preferably for 30 minutes.

The volume of solvent used to dissolve zolmitriptan in the methods of the invention will vary depending upon the amount of zolmitriptan used, the nature of the solvent, and the boiling point of the solvent. One of ordinary skill in the art with little or no experimentation can easily be determine the conditions. Typically, the volume of solvent is sufficient to dissolve or suspend the zolmitriptan at the reflux temperature of the solvent.

The volume of anti-solvent necessary to precipitate zolmitriptan will also vary depending on the amount of zolmitriptan and solvent used and the nature of the anti-solvent. One of ordinary skill in the art with little or no experimentation can easily determine the conditions. Typically, the volume of anti-solvent is sufficient to precipitate zolmitriptan at the reflux temperature of the solvent. Preferably, the ratio of anti-solvent to solvent is about 1:1 to about 1:9.

Form A may also be prepared by slurrying one of crystal form D, G, K, Q and S in an acetone/water solution (20:80).

The invention also encompasses a method of preparing zolmitriptan crystal Form A comprising: providing a solution of zolmitriptan and a solvent selected from the group consisting of a C₁-C₄ alcohol, C₅-C₈ aromatic hydrocarbon, amine, amide and tetrahydrofuran by heating to a temperature of from about 40° C. to about 100° C.; cooling to a temperature of from about 0° C. to about 25° C.; maintaining for about 12 hours to obtain a precipitate; providing a slurry of the obtained precipitate and acetone/water solution (20:80); maintaining at room temperature for about 30 minutes; cooling to a temperature of from about 0° C. to about 25° C.; maintaining for about 12 hours to obtain a precipitate and recovering zolmitriptan crystal Form A.

The method may further comprise repetition of slurring the precipitate in acetone/water solution, cooling and maintaining the slurry.

Preferably, the solvent used to form the solution is at least one of: water, isopropanol, diethylamine, tetrahydrofuran, acetonitrile, 2-butanol, n-butanol, ethanol, toluene and DMF.

Preferably, the solution is heated to the reflux temperature of the solvent.

Preferably, the solution is cooled to a temperature of about 4° C.

Preferably, the slurry is cooled to a temperature of about 4° C.

Zolmitriptan crystal Form A may then be recovered by any method known in art, such as filtration and drying the precipitate, preferably at about 60° C.-70° C. for about 12 hours at a pressure below about 100 mm Hg in a vacuum oven.

Form A may also be prepared by drying one of crystal form C, D, E, F, G, H, J, K, L, M, N, P, Q or S.

The invention also encompasses a method of preparing zolmitriptan crystal Form A comprising converting one of crystal form C, D, E, F, G, H, J, K, L, M, N, P, Q or S into Form A by drying the zolmitriptan crystal form under reduced pressure until the crystal form is substantially converted into Form A.

The term “reduced pressure” refers to a pressure less than 760 mm Hg. Preferably, Form C, D, E, F, G, H, J, K, L, M, N, P, Q or S is dried at a pressure of about less than 150 mbar to prepare Form A More preferably, at a pressure of from about 1 mbar to about 100 mbar.

The heating temperature required to convert the crystal form into Form A may be varies depending on the crystal form used to form Form A, and can be determined by reference to Example 5 and Table 3.

Preferably, the zolmitriptan crystal form is heated at a temperature of about 60° C. to about 110° C. for a time sufficient to convert the crystal into Form A.

Zolmitriptan crystal Forms D, I, G, H, I, K, J, P, and S are polymorphically stable and do not convert to Form A when maintained at room temperature.

The invention also encompasses method of preparing zolmitriptan crystal form T comprising: providing a suspension of zolmitriptan in water containing a mineral acid to obtain a reaction mixture having a pH of about 0.5 to about 1 at room temperature; adding a first inorganic base to obtain a pH of about 7; extracting with a water immiscible solvent preferably, a solvent selected from the group consisting of a C₃-C₇ ester to obtain a first two phase system; treating the first aqueous phase with charcoal; adding a second inorganic base to obtain a pH of about 11; heating the reaction mixture to a temperature of about 50° C.; extracting with a water immiscible solvent preferably, a solvent selected from the group consisting of a C₃-C₇ ester to obtain a second two phase system; combining the first and second organic phases; concentrating the combined organic phase; cooling the organic phase to obtain a precipitate and recovering zolmitriptan Form T.

Preferably, the mineral acid is selected form the group consisting of inorganic acids such as: HCl, HBr, H₃PO₄ and H₂SO₄ or an organic acid such as any carboxylic acid. Most preferably, the acid is HCL.

Preferably, the first inorganic base is an alkaline metal carbonate. More preferably the first inorganic base is selected from a group consisting of potassium carbonate and sodium carbonate. Most preferably, the base is potassium carbonate.

Preferably, the solvent used to extract the reaction mixture is ethyl acetate.

Preferably, the second inorganic base is an alkaline metal hydroxide. More preferably the first inorganic base is selected from a group consisting of potassium hydroxide and sodium hydroxide. Most preferably, the base is sodium hydroxide.

Preferably, the concentrating is by distillation.

Before concentrating, the combined organic phase may be dried with magnesium sulfate.

Preferably, the combined organic phase is cooled gradually to a temperature of about room temperature.

Zolmitriptan form T may then be recovered by any method known in art, such as filtration and drying the precipitate, preferably at about 40° C. at a pressure below about 100 mmHg in a vacuum oven.

The base is initially added to obtain a pH of about 7 in order to separate the impurities from the zolmitriptan so that the impurities are in the organic phase (ethyl acetate) and the zolmitriptan is in the aqueous phase. An additional amount of base is added later in the process to obtain a pH of about 11 in which the impurities (salts) move to the aqueous phase and the zolmitriptan moves to the organic phase (ethyl acetate).

The invention also encompasses a method of preparing amorphous form of zolmitriptan comprising: drying Form R under reduced pressure until it is converted into amorphous form.

Preferably, form R is dried under reduced pressure at a temperature of about 50° C. for a period of about 16 hours to obtain an amorphous form of zolmitriptan.

The invention also encompasses methods of preparing crystal Form J comprising drying Form O under reduced pressure until it is substantially converted into Form J.

The invention also encompasses methods of preparing crystal Form S comprising preparing Form E and then heating Form E until it is substantially converted into Form S.

The invention also encompasses methods of preparing crystal Form I comprising preparing Form F and then heating Form F until it is substantially converted into Form I.

The invention also encompasses methods of preparing crystal Form G comprising preparing Form M and then heating Form M until it is substantially converted into Form G.

The length of time necessary to substantially convert a crystal form into Form A, G, I, J and amorphous form will vary depending on the amount of starting crystal form used, and one of ordinary skill in the art will readily be able to determine that time.

The invention also encompasses a method of preparing amorphous form of zolmitriptan comprising: providing a mixture of zolmitriptan in acetonitrile at a temperature of about 50° C.; adding dichloromethane to the mixture; maintaining the mixture for about 10 minutes cooling the mixture gradually to 4° C. to obtain a precipitate and recovering amorphous form of zolmitriptan.

Amorphous form of zolmitriptan may be recovered by any method known in art, such as filtration to dryness on a funnel, preferably for 30 minutes.

In one embodiment, zolmitriptan amorphous form is prepared by a process comprising spray-drying a solution of zolmitriptan using a spray-dryer having nitrogen drying gas heated to a temperature of from about 40° C. to about 200° C. More particularly, the process comprises dissolving zolmitriptan in a polar organic solvent at about room temperature, pumping the obtained solution into a spray dryer and contacting the solution of zolmitriptan with nitrogen gas at a temperature of above from about 40° C. to about 200° C. until Amorphous form is obtained.

Polar organic solvents suitable for use in the method of the invention include, but are not limited to, C₁-C₄ alcohols, C₃-C₆ ketones and acetonitrile. Preferably, the polar organic solvent is a C₁-C₄ alcohol, and more preferably is methanol.

The volume of solvent used to dissolve zolmitriptan in the methods of the invention will vary depending upon the amount of zolmitriptan used, the nature of the solvent, and the boiling point of the solvent. One of ordinary skill in the art with little or no experimentation can easily determine a suitable volume of solvent. Typically, the volume of solvent is sufficient to dissolve or suspend the zolmitriptan at the reflux temperature of the solvent.

Amorphous form is stable upon heating and does not convert to Form A when heated at a temperature of about 60° C. to about 110° C. for a period of about 2 hours.

The invention also encompasses pharmaceutical compositions comprising at least one zolmitriptan crystal form and methods of preparing these compositions. The particle size (PS) of the active ingredient zolmitriptan crystal form is one of the key parameters of formulation of a pharmaceutical composition. The particle size of the zolmitriptan crystalline forms is up to 500 μm, preferably up to 300 μm, and more preferably up to 150 μm. Conventional methods for measuring particle size include, but are not limited to, sieves, sedimentation, electrozone sensing (coulter counter), microscopy, and Low Angle Laser Light Scattering (LALLS), may be used to determine the particle size of the zolmitriptan crystal forms. In a preferred embodiment, the zolmitriptan crystal forms have a particle size of up to about 500 μm.

The invention also encompasses methods of treating a disease condition wherein agonism of the 5-HT receptor is beneficial comprising administering an effective amount of a pharmaceutical formulation having at least one zolmitriptan crystal form to a patient in need thereof.

Having described the invention with reference to certain preferred embodiments, other embodiments will become apparent to one skilled in the art from consideration of the specification. The invention is further defined by reference to the following examples describing in detail the preparation of zolmitriptan crystal forms and methods of use of the invention. It will be apparent to those skilled in the art that many modifications, both to materials and methods, may be practiced without departing from the scope of the invention.

### EXAMPLES

Zolmitriptan crystal forms were characterized using Scintag X-ray powder diffractometer model X'TRA, Cu-tube solid state detector. The sample holder was a round standard aluminum sample holder with rough zero background quartz plate with a cavity of 25 (diameter)*O.5 mm (depth). The scanning parameters were range: 2-40 and in some cases 2-30 degrees two-theta; scan mode: continuous scan; step size: 0.05 deg.; and a rate of 3 deg/min.

Typically, to determine the Loss on Dry (LOD) by Thermal Gravimetric Analysis (TGA), a sample was heated from about 25° C. to about 200° C. at a heating rate of about 10° C. per minute, while purging with nitrogen gas at a flow rate of 40 ml/min.

Spray-drying may be performed in a conventional manner in the processes of the invention (see, e.g., Remington: The Science and Practice of Pharmacy, 19th Ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The zolmitriptan product produced by spray-drying may be recovered by techniques commonly used in the art, such as using a cyclone or a filter.

The processes of the invention are not limited to the use of any particular spray-dryer; rather, the apparatus used in the method of the invention may be any typical spray-drying apparatus. Examples of such apparatuses include Niro Models PSD-1, PSD-2 and PSD-4 (Niro A/S, Soeborg, Denmark).

### Example 1

### General Procedure for Single Solvent Crystallization

In a three necked round bottomed flask equipped with a condenser, a thermometer and a magnetic stirrer, zolmitriptan (4 g) was immersed in a volume of solvent to form a mixture. The mixture was heated at reflux or at 110° C. (the lower of the two), and maintained at the temperature for 10 minutes. The mixture was cooled gradually to 4° C. and a precipitate formed. The formed precipitate was filtered to dryness on the funnel for 30 minutes. Half of the precipitate was collected and its XRD spectrum was measured (“wet”). The other half was dried under reduced pressure at 50° C. for 16 hours and its XRD was measured (“dry”). The results are summarized in Table 2.

TABLE 2
Crystallization of zolmitriptan crystal forms by method 1

| | Volume | Temp. | Wet(w)/ | XRD |
|---|---|---|---|---|
| Solvent | (ml) | ° C. | dry(d) | Result |
| MIBK | 10 | 100 | w | Form A |
| MIBK | 10 | 100 | d | Form A |
| 2-Butanol | 10 | 100 | w | Form D |
| 2-Butanol | 10 | 100 | d | Form A |
| 1-Butanol | 10 | 100 | w | Form E |
| 1-Butanol | 10 | 100 | d | Form A |
| Isobutanol | 10 | 100 | w | Form F |
| Isobutanol | 10 | 100 | d | Form A |
| THF | 30 | 66 | w | Form G |
| THF | 30 | 66 | d | Form A |
| Cyclohexanone | 10 | 100 | w | Form H |
| Cyclohexanone | 10 | 100 | d | Form H |
| Dioxane | 10 | 100 | w | Form I |
| Dioxane | 10 | 100 | d | Form I |
| Piperidine | 10 | 100 | w | Form O |
| Piperidine | 10 | 100 | d | Form J |
| Cyclopentanone | 10 | 100 | w | Form M |
| Cyclopentanone | 10 | 100 | d | Form A |
| Methylethylketone | 20 | 80 | w | Form N |
| Methylethylketone | 20 | 80 | d | Form A |
| Pyridine | 10 | 100 | w | Form P |
| Pyridine | 10 | 100 | d | Form A + I |
| Diethylamine | 55 | 60 | w | Form Q |
| Diethylamine | 55 | 60 | d | Form A |
| Dichloromethane | 40 | 42 | w | Form R |
| Dichloromethane | 40 | 42 | d | Form Amorphous |
| Butylacetate | 110 | 125 | w | Form S |
| Butylacetate | 110 | 125 | d | Form S |

### Example 2

### General Procedure for Solvent-Anti-Solvent System

In a three necked round bottomed flask equipped with a condenser, a thermometer and a magnetic stirrer, zolmitriptan (4 g) was dissolved in a volume of solvent at reflux condition or at 100° C. (the lower of the two) to form a mixture. An anti-solvent is added to the mixture while heating until a precipitate forms. After 10 minutes the mixture is cooled gradually to 4° C. The precipitate was filtered to dryness on a funnel for 30 minutes. Half of the precipitate was collected and its XRD spectrum was measured (“wet”). The other half was dried under reduced pressure at 50° C. for 16 hours and its XRD was measured (“dry”). The results are summarized in Table 3.

TABLE 3
Crystallization of zolmitriptan crystal forms by method 2

| | Volume | Temp. | | Volume | | |
|---|---|---|---|---|---|---|
| Solvent | (ml) | ° C. | Anti-Solvent | (ml) | Wet(w)/dry(d) | Result |
| DMSO | 10 | 100 | Toluene | 80 | w | Form A¹ |
| DMSO | 10 | 100 | Toluene | 80 | d | Form A |
| DMF | 10 | 100 | Cyclohexane | 90 | w | Form B |
| DMF | 10 | 100 | Cyclohexane | 90 | d | Form A |
| Ethanol | 10 | 78 | P.E. (40-60) | 10 | w | Form C |
| Ethanol | 10 | 78 | P.E. (40-60) | 10 | d | Form A |
| Acetonitrile | 10 | 81 | Cyclohexane | 15 | w | Form J² |
| Acetonitrile | 10 | 81 | Cyclohexane | 15 | d | Form A |
| DMF | 10 | 100 | Xylenes | 90 | w | Form K³ |
| DMF | 10 | 100 | Xylenes | 90 | d | Form A |
| Acetonitrile | 10 | 100 | Toluene | 15 | w | Form K |
| Acetonitrile | 10 | 100 | Toluene | 15 | d | Form A |
| Acetonitrile | 10 | 81 | Chlorobenzene | 15 | w | Form L |
| Acetonitrile | 10 | 81 | Chlorobenzene | 15 | d | Form A |
| Acetonitrile | 10 | 81 | Dichloromethane | 90 | w | Form A + P |
| Acetonitrile | 10 | 81 | Dichloromethane | 90 | d | Form Amorphous |

¹Additional peaks were observed in the XRD diffractogram at: 13.3, 17.1, and 23.5.
²Additional peaks were observed in the XRD diffractogram at 19.6, 22.1, and 23.3.
³An additional peak was observed in the XRD diffractogram at 19.3.

### Example 3

### Preparation of Amorphous Zolmitriptan by Spray Dryer

Zolmitriptan (5 g) was dissolved in methanol (40 ml) at room temperature. The solution obtained was pumped into a spray dryer at a feed rate of 2 ml/minute and contacted with nitrogen gas. The nitrogen gas was at an inlet temperature of 100° C. The evaporated solvent and nitrogen left the spray dryer at a temperature of 64° C.

### Example 4

### Preparation of Amorphous Form by Spray Dryer

Zolmitriptan (5g) was dissolved in methanol (40 ml) at room temperature. The solution obtained was pumped into the spray dryer at a feed rate of 2 ml/minute. The nitrogen was at an inlet temperature of 50° C. The evaporated solvent and nitrogen exited the spray dryer at 34° C.

### Example 5

### General Procedure for Transformation by Heating

Zolmitriptan (0.5 g) crystalline forms were heated in a conventional oven for a period of about 1.5 to 2 hours at different temperatures. The crystal form of the samples was determined by XRD before and after heating. The results are summarized in Table 4.

TABLE 4
Transformation of zolmitriptan by heating

| | | Crystal | | Crystal | | Crystal |
|---|---|---|---|---|---|---|
| | Heating | form | Heating | form | Heating | form |
| Initial | temp. | after | temp. | after | temp. | after |
| Form | [° C.] | heating | [° C.] | heating | [° C.] | heating |
| A | 90 | A | 110 | A | | |
| C | 60 | A | | | | |
| D | 60 | D | 80 | A +˜ 5% D | 120 | A |
| E | 60 | S | 90 | A | | |
| F | 60 | I | 90 | A | | |
| G | 60 | G | 90 | A | | |
| H | 60 | H | 90 | H + 15% A | 110 | A |
| I | 60 | I | 100 | A | | |
| K | 60 | K | 100 | A | | |
| L | 90 | A + | 110 | A + 20% L | | |
| | | 20% L | | | | |
| M | 60 | G | 90 | A + 10% G | | |
| N | 60 | N + | 90 | A | | |
| | | A | | | | |
| J | 60 | J | 90 | J + 30% A | | |
| P | 60 | P | 90 | A + 20% P | 110 | A |
| Q | 60 | A + | 90 | A | 110 | A |
| | | 10% Q | | | | |
| S | 60 | S | 100 | A +˜ 5% S | 110 | A |
| Amorph | 60 | A- | 100 | Amorph | 110 | Amorph |
| | | morph | | | | |

### Example 6

### General Procedure for Transformation by Heating and Slurry in Acetone-Water Mixture

A sample of zolmitriptan (6 g) was placed in a glass flask with solvent and heated to reflux until the zolmitriptan dissolved. The zolmitriptan was cooled to room temperature while stirring, and left at 4° C. overnight. The mixture was filtered and the solid obtained was divided in half. One half of the solid was labeled “wet” and the other half was dried in a vacuum oven at about 60°-70° C. overnight and labeled as “dried.”

One third of the wet portion of each sample was separated for XRD, and marked as “_w”. One third of the dried portion of each sample was separated for XRD, and marked as “_d”. For example, “1w” refers to the wet portion of sample 1 analyzed by XRD before slurry in acetone/water, and “1d” refers to the dried portion of sample 1 analyzed by XRD before slurry in acetone/water.

The remaining two-thirds of the wet sample was weighed, placed in a glass flask containing 10 v/w of acetone:water (20:80), stirred for about 30 minutes at room temperature, and left at about 4° C. overnight. The mixture was filtered and the solid obtained was divided in half. One half was labeled “_w-ac-w.” The other half was dried in a vacuum oven at about 60° C.-70° C. overnight, and labeled “_w-ac-d.” For example, “1w-ac-w” refers to the portion of the wet sample resulting from slurry in acetone/water, while “1w-ac-d” refers to the portion of the wet sample resulting from slurry in acetone/water that was dried.

Likewise, the remaining two-thirds of the dry sample was weighed, placed in a glass flask containing 10 v/w of acetone:water (20:80), stirred for about 30 minutes at room temperature, and left at about 4° C. overnight. The mixture was filtered and the solid obtained was divided in half. One half was labeled “_d-ac-w.” The other half was dried in a vacuum oven at about 60° C.-70° C. overnight, and labeled “_d-ac-d.” For example, “1d-ac-w” refers to the portion of the dry sample resulting from slurry in acetone/water, while “1d-ac-d” refers to the portion of the dry sample resulting from slurry in acetone/water that was then dried.

Zolmitripan Samples:

Sample 1 was dissolved in 15 ml of isopropanol: water 9:1.

Sample 2 was dissolved in 120 ml of diethylamine.

Sample 3 was dissolved in 45 ml of tetrahydrofuran (THF).

Sample 4 was dissolved in 15 ml of acetonitrile (CAN).

Sample 5 was dissolved in 15 ml of 2-butanol.

Sample 6 was dissolved in 15 ml of n-butanol.

Sample 7 was dissolved in 15 ml of ethanol and 15 ml toluene was added.

Sample 8 was dissolved in 120 ml of DMF: toluene 1:19.

The results are summarized in Table 5.

TABLE 5
Transformation by heating/slurry

| Sample | Resulting Crystal Form |
|---|---|
| 1w | Form A |
| 1w-ac-w | Form A |
| 1w-ac-d | Form A |
| 1d | Form A |
| 1d-ac-w | Form A |
| 1d-ac-d | Form A |
| 2w | Form Q |
| 2w-ac-w | Form A |
| 2w-ac-d | Form A |
| 2d | Form A |
| 2d-ac-w | Form A |
| 2d-ac-d | Form A |
| 3w | Form G |
| 3w-ac-w | Form A |
| 3w-ac-d | Form A |
| 3d | Form A |
| 3d-ac-w | Form A |
| 3d-ac-d | Form A |
| 4w | Form A |
| 4w-ac-w | Form A |
| 4w-ac-d | Form A |
| 4d | Form A |
| 4d-ac-w | Form A |
| 4d-ac-d | Form A |
| 5w | Form D |
| 5w-ac-w | Form A |
| 5w-ac-d | Form A |
| 5d | Form A |
| 5d-ac-w | Form A |
| 5d-ac-d | Form A |
| 6w | Form S |
| 6w-ac-w | Form A |
| 6w-ac-d | Form A |
| 6d | Form A |
| 6d-ac-w | Form A |
| 6d-ac-d | Form A |
| 7w | Form K |
| 7w-ac-w | Form K +˜ 30% A |
| 7w-ac-d | Form A |
| 7d | Form A |
| 7d-ac-w | Form A |
| 7d-ac-d | Form A |
| 8w | Form K |
| 8w-ac-w | Form K +˜ 30% A |
| 8w-ac-d | Form A |
| 8d | Form A |
| 8d-ac-w | Form A |
| 8d-ac-d | Form A |

### Example 6a

### General Procedure for Transformation by Heating and Slurry in Other Solvents or Mixtures

Similarly to example 6 Zolmitriptan samples were slurried from other solvents or solvent systems after dissolution and precipitation from a specific solvent. The results are summarized in table 5A:

TABLE 5A
Transformation by heating/slurry

| Crystallized | | | |
|---|---|---|---|
| from | Form | Treatment * | XRD sample |
| DCM | R | n-Hexane | Form D |
| DCM | R | n-Hexane, dried 63° C. | Essent. Am D |
| Butyl acetate | S | n-Hexane | Form D |
| Butyl acetate | S | n-Hexane, dried 63° C. | Form A +˜ 20% D |
| 1-Butanol | E | n-Hexane | Form D |
| 1-Butanol | E | n-Hexane, dried 63° C. | Form A +˜ 30% D |
| THF | G | n-Hexane | Form D |
| THF | G | n-Hexane, dried 63° C. | Essent. Am D |
| MEK | N | n-Hexane | Form D |
| MEK | N | n-Hexane, dried 63° C. | Form A |
| 1,4-Dioxane | I | n-Hexane | Form D |
| 1,4-Dioxane | I | n-Hexane, dried 63° C. | Form A |
| Ethanol/Toluene | K | n-Hexane | Form D |
| Ethanol/Toluene | K | n-Hexane, dried 63° C. | Form A |
| DCM | R | Toluene | Form K |
| DCM | R | Toluene, dried 63° C. | Essent. Am K + A |
| Butyl acetate | S | Toluene | Form K |
| Butyl acetate | S | Toluene, dried 63° C. | Form A +˜ 10% K |
| 1-Butanol | E | Toluene | Form K |
| 1-Butanol | E | Toluene, dried 63° C. | Form K |
| THF | G | Toluene | Form K |
| THF | G | Toluene, dried 63° C. | Form A +˜ 10% K |
| MEK | N | Toluene | Form K |
| MEK | N | Toluene, dried 63° C. | Form A |
| 1,4-Dioxane | I | Toluene | Form K |
| 1,4-Dioxane | I | Toluene, dried 63° C. | Form A |
| Ethanol/Toluene | K | Toluene | Form K |
| Ethanol/Toluene | K | Toluene, dried 63° C. | Form A |
| DCM | R | Water | Form A |
| DCM | R | Water, dried 63° C. | Form A |
| Butyl acetate | S | Water | Form A +˜ 10% D |
| Butyl acetate | S | Water, dried 63° C. | Form A |
| 1-Butanol | E | Water | Form A |
| 1-Butanol | E | Water, dried 63° C. | Form A |
| THF | G | Water | Form A |
| THF | G | Water, dried 63° C. | Form A |
| MEK | N | Water | Form A |
| MEK | N | Water, dried 63° C. | Form A |
| 1,4-Dioxane | I | Water | Form A |
| 1,4-Dioxane | I | Water, dried 63° C. | Form A |
| Ethanol/Toluene | K | Water | Form A +˜ 10% K |
| Ethanol/Toluene | K | Water, dried 63° C. | Form A |

* solid was immersed in 10 v/w of solvent, filtered, half was sucked for ½ hour on Buchner, other half was dried afterwards under reduced pressure.

### Example 7

### Preparation Zolmitriptan Form T

Zolmitriptan (18 g) was suspended in 660 ml water containing 73 ml HCl to obtain a pH of 0.5-1 at room temperature. The reaction mixture was then brought to pH 7 by addition of 18 g of K₂CO₃ and afterwards was extracted twice with 200 ml of ethylacetate. The aqueous phase was treated with 1 g of charcoal (Norit SX) for 1 hour. Afterwards, the charcoal was filtered. The pH of the aqueous phase was brought to 11 with 20 ml NaOH solution (20%) followed by heating to 50° C. The aqueous phase was extracted 3 times with 300 ml of ethyl acetate at 50° C. The combined organic phase was dried with anhydrous magnesium sulfate. The organic phase was distilled to the volume of 140-160. The obtained solution was cooled slowly to room temperature, and left to crystallize overnight. The substance obtained was filtered. 15 g of the wet (14% wetness) substance was obtained. The filtrate was dried at 40° C., under reduced pressure. 12.9 r of crude Zolmitriptan (76.5% assay) was obtained.

1-38. (canceled)

39. A zolmitriptan crystal Amorphous form characterized by an XRD pattern substantially identified by FIG. 19.

40-49. (canceled)

50. A pharmaceutical composition comprising the zolmitriptan of claim 39 and a pharmaceutically acceptable excipient.

51. A method of making a pharmaceutical composition comprising mixing the zolmitriptan of claim 39 and at least one pharmaceutically acceptable excipient.

52. A method of treating a disease condition wherein agonism of the 5-HT receptor is beneficial comprising administering a therapeutically effective amount of the zolmitriptan of claim 39 to a patient in need thereof.

53. The zolmitriptan crystal according to claim 39, wherein the zolmitriptan crystal has about 0% to about 3% water by weight.

54. The zolmitriptan crystal according to claim 39, wherein the zolimitriptan crystal has a weight loss as measured by TGA of about 0% to about 3% by weight.

55. A method of preparing a zolmitriptan crystal Amorphous form characterized an XRD pattern substantially identified by FIG. 19 comprising:
a) providing a zolmitriptan crystal Form R; and
b) drying the zolmitriptan crystal Form R under reduced pressure to obtain zolmitriptan crystal Amorphous form.

56. The method according to claim 60, wherein the drying step is carried out at a temperature of about 50° C. for about 16 hours.
